(19) United States
(12) Patent Application Publication
(10) Pub. No.: US 2013/0143805 A1
(43) Pub. Date: Jun. 6, 2013

(54) GGF2 AND METHODS OF USE

(71) Applicants: Matthew Whittaker, Washington, DC (US); Jean R. Wrathall, Arlington, VA (US)

(72) Inventors: Matthew Whittaker, Washington, DC (US); Jean R. Wrathall, Arlington, VA (US)

(73) Assignee: GEORGETOWN UNIVERSITY, Washington, DC (US)

(21) Appl. No.: 13/816,779

(22) PCT Filed: Aug. 12, 2011
(86) PCT No.: PCT/US11/47606

§ 371 Date: Feb. 13, 2013

Related U.S. Application Data
(60) Provisional application No. 61/373,541, filed on Aug. 13, 2010.
(60) Provisional application No. 61/374,777, filed on Aug. 18, 2010.
(60) Provisional application No. 61/413,768, filed on Nov. 15, 2010.

(51) Int. Cl. A61K 38/18

(52) U.S. Cl. CPC A61K 38/18
USPC 514/7.6; 435/375

(57) ABSTRACT

Provided herein are methods of treating central nervous system injury (e.g., spinal cord injury) using GGF2 and compositions comprising GGF2. For example, provided is a method of treating spinal cord injury in a subject, comprising administering at least one dosage of less than 1 mg/kg of GGF2 to the subject. Also provided are methods of promoting proliferation of neural stem cells and of promoting revascularization comprising using GGF2 and compositions comprising GGF2.

### CROSS-REFERENCE TO RELATED APPLICATIONS

This application claims the benefit of U.S. Provisional Application No. 61/373,541, filed on Aug. 13, 2010, U.S. Provisional Application No. 61/374,777, filed Aug. 18, 2010, and U.S. Provisional Application No. 61/413,768, filed on Nov. 15, 2010, which are incorporated by reference herein in their entireties.

### STATEMENT REGARDING FEDERALLY FUNDED RESEARCH

This invention was made with government support under grants ROI-NS35647 and T32-NS041218 awarded by the National Institutes of Health. The government has certain rights in the invention.

### SUMMARY

Provided herein are methods of treating spinal cord injury using GGF2 and compositions comprising GGF2. For example, provided is a method of treating spinal cord injury in a subject, comprising administering at least one dosage of less than 1 mg/kg of GGF2 to the subject. Also provided are methods of promoting proliferation of glial precursor cells comprising contacting the glial precursor cells with GGF2 and methods of promoting revascularization of neural tissue following central nervous system injury in a subject comprising administering to the subject GGF2.

### BACKGROUND

Spinal cord injury affects approximately 11,000 new individuals each year in the United States. The majority of these cases are contusion injuries, where pressure from the vertebral bones crushes the spinal cord, causing immediate damage to neural cells and fiber tracts. Other types of central nervous system injury or damage include traumatic brain injury, stroke, and other types of acquired brain injury (e.g., caused by disease or surgery). About 1.7 million people sustain a traumatic brain injury annually in the United States. This “primary injury” triggers the onset of the hypoxia and inflammation that cause “secondary injury,” a progressive destruction of neurons and glial, as well as fiber tracts passing through the injury site.

### BRIEF DESCRIPTION OF THE DRAWINGS

FIG. 1 shows an outline of experimental design. Animals received a moderate contusive spinal cord injury at T8-T9. Drug treatments were administered once daily, for seven days, beginning 1 day post injury. BrdU (17 mg/kg) was administered on days 2, 3, and 4 in rat studies, and on days 2, 4, and 7 in mouse studies. Functional recovery was determined once weekly according to the combined behavioral score (CBS) (Gale et al., Exp. Neurol. 88:123-34 (1985)) and Basso, Beattie, and Bresnahan (BBB) (Basso et al., J. Neurotrauma 12:1-21 (1995)) scores in rats and the Basso Mouse Scale (BMS) for locomotion in mice (Basso et al., Exp. Neurol. 139:244-56 (2006).

FIGS. 2A-2C show phenotypic distribution of cells labeled with BrdU after SCI in the rat. Counts were made in the ventromedial white matter (VMWM) at locations 2, 3, and 4 mm rostral and caudal to the injury epicenter at 7 days after spinal cord injury (SCI). FIG. 2A shows a histogram demonstrating that a 1 week treatment with GGF2 after SCI increased endogenous cell proliferation. This effect was most pronounced in sections 2 mm from the epicenter. FIG. 2B shows a histogram demonstrating that NG2⁺ precursors constitute approximately one half of the total BrdU⁺ cells in both GGF2 and saline treated rats. FIG. 2C shows a histogram demonstrating that GGF2 treatment does not influence the number of BrdU⁺/OX42⁺ cells in VMWM. *Significant difference between treatment groups; GGF2; n=8; saline: n=8, two way repeated measures ANOVA, Tukey's HSD, p<0.05.

FIGS. 3A-3B show functional recovery after SCI in GGF2 vs. saline treated rats. Both BBB (FIG. 3A) and CBS (FIG. 3B) behavioral tests show greater functional recovery in GGF2 treated rats. The BBB (0=paralysis; 21=normal), which evaluates open field locomotion, showed a difference in recovery by the second week after SCI. The CBS (100=paralysis; 0=normal), which evaluates overall hind limb sensory-motor deficits, indicated a significant reduction in functional deficits in the GGF2 treated group by the fourth week after injury. *Significant difference between groups at indicated time point; n=11 per group, two way repeated measures ANOVA, Tukey's HSD, p<0.05.

FIGS. 4A-4C show histological comparison of spinal cords from GGF2 vs. saline treated rats at 7 days (n=4/group) and 42 days (n=8/group) post injury. FIG. 4A shows a histogram demonstrating that at 7 days, both treatment groups show similar white matter areas at all locations tested. FIG. 4B shows a histogram demonstrating that at 42 days, GGF2 treated subjects display more white matter than saline treated at the epicenter and 1 mm rostral and caudal to the epicenter. *p<0.05 vs. saline, two way repeated measures ANOVA, Tukey's HSD. FIG. 4C shows an image of tracings of eriochrome staining at the injury epicenters of spinal cords from saline (top panel) vs. GGF2 (bottom panel) treated rats demonstrating greater white matter sparing in the GGF2 treated group.

FIG. 5 shows a graph demonstrating that treatment with systemic GGF2 or FGF2+GGF2 improves functional recovery from incomplete spinal cord injury in CNP-EGFP mice. Bars represent mean±SEM. Two way repeated measures ANOVA, Tukey's HSD, *p<0.05; **p<0.001 vs. saline control.

FIGS. 6A-6E show confocal images of residual WM at the injury epicenter of a GGF2 treated CNP-EGFP transgenic mouse at 7 d post injury. FIGS. 6A-6C show immunohistochemical staining of sections fluorescently labeled for NG2 and CC1. The images were captured at 60×. Left-pointing arrows: EGFP⁻/NG2⁺ cells (FIGS. 6A and 6C), Right-pointing arrow: EGFP⁺/NG2⁺ cell (FIG. 6C), Vertical-pointing arrows: EGFP⁺/CC1⁺ cells (FIGS. 6B and 6C). Scale bar=20 μm. FIG. 6D shows an eriochrome-cyanine stained image of a representative injury epicenter demonstrating the locations of cell counting. Cells were counted within an ROI (region of interest) of 0.02 mm² (grey boxes) in the left and right ventral-lateral areas of spared white matter at the injury epicenter and sections 200 μm rostral and caudal to the epicenter in saline treated and GGF2 treated subjects. Scale bar=500 μm. FIG. 6E shows a histogram demonstrating that GGF2 treatment increased the total number of NG2⁺ cells as well as non-oligodendrocyte lineage NG2 cells (EGFP⁻/NG2⁺), and total oligodendrocyte lineage cells (EGFP⁺) at 7 days post injury. *p<0.05 One way ANOVA, Bonferonni post hoc test.

FIGS. 7A-7C show that one week treatment with systemic GGF2 increases the number of Sox2⁺/EGFP⁺ cells at the injury epicenter at 7 days post injury in CNP-EGFP transgenic mice.

FIG. 7A shows a representative image from spared white matter (WM) of a GGF2 treated mouse at 7 days post injury. Arrow: Sox2⁺/EGFP⁺ cell. Scale bar=20 μm. FIGS. 7B and 7C show histograms of cells counted in both spared WM and non-WM at the injury epicenter and sections 200 μm rostral and caudal to the epicenter in saline treated and GGF2 treated subjects. GGF2 treatment did not significantly influence the total number of cells expressing Sox2 (FIG. 7B), but increased the number of oligodendrocyte lineage cells that expressed Sox2 at 7 days post injury (FIG. 7C). Bars represent mean±SEM. Values in parentheses indicate number of subjects. *p<0.05 vs. saline control, Student's t test.

FIGS. 8A-8C show that one week treatment with systemic GGF2 or FGF2+GGF2 increases the number of mature oligodendrocytes in spared WM at the injury epicenter at 28 days post injury in CNP-EGFP mice. CC1⁺ cells were counted via unbiased stereology at the injury epicenter and at 200 μm rostral and caudal to the epicenter. FIG. 8A shows a representative image of spared WM at injury epicenter of GGF2 treated subject. Arrows indicate CC1⁺ cells. FIG. 8B shows a histogram demonstrating that treatment with GGF2 alone or FGF2+GGF2 increases mature oligodendrocytes at the epicenter in residual WM. FIG. 8C shows a histogram demonstrating that no effect of any drug treatment is seen on mature oligodendrocyte number in non-WM at the epicenter. Bars represent mean±SEM. *p<0.05; **p<0.001 vs. saline control, one-way ANOVA, Tukey HSD. FIGS. 8D-8F show that one week GGF2 treatment increases the number of mature oligodendrocytes derived from cells that were dividing during GGF2 treatment. Subjects received BrdU injections on days 2, 4, and 7 after injury. CC1⁺/BrdU⁺ cells were counted at the injury epicenter and at 200 μm rostral and caudal to the epicenter at 28 d post injury. FIG. 8D shows a representative image of spared WM at the injury epicenter of GGF2 treated subject showing CC1 and BrdU immunostaining. Arrows indicate CC1⁺/BrdU⁺ cells. FIG. 8E shows a histogram demonstrating that GGF2 treatment increases total CC1⁺/BrdU⁺ cells. FIG. 8F shows a histogram demonstratring that GGF2 treatment increases the percentage of total mature oligodendrocytes at 28 days post injury that are derived from cells that were proliferating in the first week after SCI. Bars represent mean±SEM. Values in parentheses indicate number of subjects. **p<0.01 vs. saline control, Student's t test.

FIGS. 9A-9I show that GGF2 treatment does not affect spared WM area, PLP percentage area, or NF200⁺ axon number at the injury epicenter at 28 days post injury in CNP-EGFP mice. FIG. 9A shows a representative image of eriochrome staining that was carried out on a spinal cord section at 28 days post injury to quantify residual white matter. Scale bar: 100 μm. The images were taken at 2.5× magnification, and analyzed using NIH ImageJ software. The threshold was set to display eriochrome-cyanine positive pixels based on the gray values of the digital image. FIGS. 9B and 9C show histograms demonstrating that there was no significant effect of drug treatment on WM area at any of the locations examined (One way ANOVA) at 7 days (FIG. 9B) or 28 days (FIG. 9C) post injury. Horizontal lines indicate the range of mean WM area values for uninjured subjects (n=5). FIG. 9D shows PLP staining (marker for central nervous system myelin) in a section adjacent to the eriochrome stained section in FIG. 9A. PLP immunofluorescent staining was measured at the injury epicenter and at points 200 μm rostral and caudal to the epicenter. FIGS. 9E and 9F are histograms demonstrating that GGF2 treatment did not affect the percent area of PLP staining in white matter (FIG. 9E) or non white matter (FIG. 9F) compared to saline treated controls. (One way ANOVA, Tukey HSD). FIG. 9G shows NF200 staining in a section adjacent to the eriochrome stained section in FIG. 9A. NF200 immunofluorescent staining was measured at the injury epicenter and at points 200 μm rostral and caudal to the epicenter. FIGS. 9H and 9I are histograms demonstrating that GGF2 treatment did not affect the number of NF200⁺ axons present in white matter (FIG. 9H) or non white matter (FIG. 9I) compared to saline treated controls. (One way ANOVA, Tukey HSD). Values in parentheses indicate number of subjects.

FIGS. 10A-10F show that GGF2 treatment increases Schwann cell myelination of the injury site at 28 days post injury in CNP-EGFP mice. Cells were counted in both spared WM and non-WM at the injury epicenter and sections 200 μm rostral and caudal to the epicenter in saline treated and GGF2 treated subjects. FIG. 10A is a 20× tilescan of the injury epicenter from a GGF2 treated subject at 28 days post injury showing P0 (marker of peripheral nervous system myelin, Far Left), CNP-EGFP (Middle Left), and NF200 (Middle Right) staining in separate panels. The far right panel shows a merge of the P0, CNP-EGFP, and NF200 panels. FIG. 10B shows a higher power magnification of the box located in the lesion of the merged image in FIG. 10A showing P0 myelinated axons within the lesion. FIG. 10C shows a higher power magnification of the box located dorsally in the merged image in FIG. 10A showing P0 myelinated axons in the dorsal WM. FIG. 10D shows a histogram demonstrating that GGF2 treatment increases total P0 staining at 28 days post injury vs. saline control. GGF2 treatment also increases P0 staining within the lesion (FIG. 10E) and in residual WM (FIG. 10F). Bars represent mean±SEM. Values in parentheses indicate number of subjects. *p<0.05; **p<0.001 vs. saline control, Student's t test. Scale bar: 50 μm.

FIG. 11 shows GGF2 treatment increases the number of pericytes within the lesion site at 7 days post injury. Spinal cord sections from 7 days post injury NG2-dsRed×CNP-EGFP double transgenic mice were labeled with antibodies against markers for blood vessels (Rat anti-CD31) as well as pericytes (Rb anti-PDGFRβ). Images were captured at 40× using an Olympus FV300 laser scanning confocal microscope. For each subject, pericytes were counted in 2 separate 0.03 mm² regions of interest within the lesion. Pericytes were characterized as cells that were NG2⁺/PDGFRβ⁺ and directly opposed to CD31⁺ blood vessels. Pericytes were also counted at the lesion border and in spared ventrolateral white matter. GGF2 had no effect on pericyte number in these regions.

FIG. 12 shows that GGF2 treatment increases the amount of CD31⁺ staining, a measure of revascularization, within the lesion site at 7 days post injury. Spinal cord sections from 7 days post injury NG2-dsRed×CNP-EGFP double transgenic mice were labeled with an antibody against the blood vessel marker CD31 (Rat anti-CD31). For each subject, total CD31⁺ pixels in 2 separate 0.03 mm² regions of interest within the lesion were quantified using NIH ImageJ software. CD31⁺ staining was also quantified at the lesion border and in spared ventrolateral white matter. GGF2 had no effect on CD31⁺ staining in these regions.

FIG. 13 shows that GGF2 treatment does not affect the number of p75⁺ Schwann cell precursors in non-white matter near the injury epicenter at 28 days post injury. GGF2 also had no effect on overall p75⁺ staining (white matter+non-white matter) or in white matter alone. Spinal cord sections from the epicenter +/−0.8 mm of injured CNP-EGFP mice were stained with Rb anti-p75 antibody. 20× tilescan images of entire spinal cord sections were taken using a Zeiss 510 LSM laser scanning confocal microscope. p75⁺ pixels were quantified using NIH ImageJ software. GGF2 increases peripheral myelin (P0 staining) at the epicenter within the lesion (FIG. 10) at 28 days post injury. Without intending to be limited by theory, the lack of an effect of GGF2 on p75 staining suggests that GGF2 may act by increasing the amount of myelin produced by existing Schwann cells rather than by stimulating the proliferation of new Schwann cells.

### DETAILED DESCRIPTION

Demyelination and abnormal remyelination of axons are major pathological consequences of chronic spinal cord injury (SCI) and brain injury. Axons lacking proper myelination are unable to efficiently conduct action potentials. The adult spinal cord, for example, contains a pool of endogenous glial precursor cells which spontaneously respond to SCI with increased proliferation. As used throughout, spinal cord injury is used by way of example. The same methods are useful in all central nervous system injuries.

In experimental models of SCI, most of the grey matter is destroyed within 24 hours of the initial injury inducing impact, resulting in a central hemorrhagic lesion. By 6 weeks after SCI, a large cavity forms in the place of previous grey matter, flanked by a thin rim of residual white matter. Tissue sparing is directly related to injury severity, where milder impacts cause incomplete injuries, where the rim of spared white matter is thicker, and the subjects retains some sensory and motor function below the injury site. A more severe impact causes complete injury, where all ascending and descending pathways are destroyed, and no function remains caudal to the lesion.

By 24 hours after SCI, 50% of the oligodendrocytes and astrocytes in the spared residual white matter at the epicenter are lost, contributing to early white matter pathology. Chronically after injury, the remaining axons at the injury site are sheathed with poorly compacted, thin myelin that leaves large peri-axonal spaces. Pathology is particularly severe in larger diameter axons that rely most heavily on salutatory conduction for signal propagation. The resulting axonal functional impairment can lead to further axonal and neural degeneration through reduced activity dependent trophic support.

Sparing oligodendrocytes through treatment with the AMP-A/kainate glutamate receptor antagonist NBQX, significantly reduces acute white matter pathology as well as chronic white matter loss and functional deficits. The loss of oligodendrocytes can lead to neuronal cell death and axonal collapse. Further, transplantation of oligodendrocytes has been shown to increase tissue sparing and significantly improve functional recovery after spinal cord contusion.

Loss of astrocytes and the important functions served by them also contribute to pathology after SCI. By maintaining ionic homeostasis and reducing extracellular glutamate levels, astrocytes can reduce the span of lesion progression. Astrocytes also secrete growth factors that ameliorate injury through neuroprotection, induce glial proliferation, and promote myelination.

Transplantation of astrocytes into demyelinated spinal cords has improved the ability of host oligodendrocytes to remyelinate white matter tracts. Data from transplantation studies indicate that increasing astrocyte and oligodendrocyte numbers after SCI improve functional recovery. While transplantation is a strategy that has been used to successfully introduce new cells into the damaged rodent CNS, there are recognized problems with this approach for clinical applications. Surgical manipulation of the fragile post-injury spinal cord can result in further complications (e.g., mechanical damage to the cord, infection, and/or hemorrhage). Graft-host incompatibility can be a problem, particularly in the injured cord where the blood-brain barrier is compromised. In addition, there are ethical and legal concerns regarding the sources of appropriate tissue for transplantation. An attractive alternative to transplantation is to stimulate proliferation of endogenous precursors to yield functional mature glial that proliferate following SCI.

In experimental demyelination, retrovirus-marked endogenous precursor cells in the adult rat spinal cord have been shown to remyelinate axons. The normal adult central nervous system contains its own pool of glial progenitors that can proliferate and differentiate into a number of neural phenotypes in vitro, and mature oligodendrocytes in vivo. These cells label with an antibody for the NG2 condroitin sulfate proteoglycan, and are easily distinguished by their elongated shape and small cell body that is mostly filled with a nucleus. BrdU labeling has been used to show that in the intact rat spinal cord, these cells divide and produce colonies.

While they express NG2 within 24 hours of BrdU labeling, by 4 weeks they differentiate into mature oligodendrocytes. Despite the loss of 50% of local oligodendrocytes and astrocytes by 24 hours in models of SCI, the densities of mature oligodendrocytes and astrocytes return to normal or near normal levels by 6 weeks after injury.

Recovery of cell densities is due, in part, to the proliferation of the glial progenitor cells. Following ischemic stroke injury, the density of glial progenitors begins to increase by 2 days, and this increase is accompanied by a restoration of oligodendrocyte and myelin density as early as 2 weeks. Proliferation of NG2+ cells occurs from 1 day through 8 weeks after SCI. The rise detected in the numbers of BrdU+/NG2+ cells is associated with a three-fold increase one week later in the numbers of CCI+ oligodendrocytes, showing that these progenitors are a major source of cell renewal in the injured central nervous system, e.g., the spinal cord. Endogenous progenitors and their progeny may be a part of the endogenous recovery mechanisms that help the chronic repopulation of the injured spinal cord.

GGF2 is used to stimulate the proliferation of glial progenitors following spinal cord injury in vivo. GGF2 stimulates proliferation of glial progenitors in vitro and its levels are increased significantly in the week after injury to the CNS, when glial proliferation begins.

GGF is a member of the neuregulin family of proteins, which are alternatively spliced from the NRG-1 gene. First studied for its ability to promote proliferation and differentiation of glial cells—and thus named glial growth factor—NRG-1 has since been labeled under other names such as NDF, ARIA, and heregulin. GGF triggers the proliferation of glial progenitors, and induces a phenotypic reversion in cultured oligodendrocytes, causing their return to a mitotic state. In cultured glial progenitors, GGF promotes survival and stimulates proliferation, while maintaining the cells in an immature phenotype.

Levels of GGF/NRG have been shown to increase in patients with multiple sclerosis (MS) and in cortical incision injury. ErbB receptor levels (family of GGF receptors) also increase following closed head injury, cortical incision injury, axotomyinduced Wallerian degeneration, and multiple sclerosis (MS). In a chronic relapsing model for MS, exogenously administered GGF/NRG protein can delay relapse, reducing autoimmune demyelination and promoting remyelination. GGF2 is a strong mitogen for glial progenitors that help sheathe demyelinated axons.

Traumatic spinal cord injury (SCI) leads to permanent loss of sensory and motor function caudal to the injury site. While the initial impact destroys many local neurons and glial, cell loss is not limited to the primary mechanical insult, but is exacerbated by secondary mechanisms. About 50% of the oligodendrocytes and astrocytes in the spared residual white matter of the epicenter are lost by 24 hours. The loss of astrocytes can contribute to abnormal ionic homeostasis, while oligodendrocyte loss leads to poor myelination—as seen in multiple sclerosis—and thus hindered axonal transmission.

Despite this initial cell loss, oligodendrocyte and astrocyte densities in the residual white matter return to control levels by 6 weeks after SCI. Recovery of cell densities is due in part to the proliferation of surviving glial cells. Bromodeoxyuridine (BrdU) labeling studies show that proliferation of cells within 4 mm of the epicenter is significantly upregulated in the week following SCI. These BrdU labeled cells can be detected at 6 weeks following SCI, and comprise approximately one sixth of CCI+mature oligodendrocytes.

GGF2, a mitogen for glial progenitors in vitro, is upregulated following injury. GGF2 is developmentally involved in the axonal regulation of oligodendrocyte and Schwann cell expansion and subsequent myelination. GGF2 is a strong mitogen for glial precursors and oligodendrocytes, and, following injury, ligand and receptor (erbB receptors) levels are upregulated at the injury site. Three days after SCI, GGF2 mRNA is significantly elevated rostrally. Addition of recombinant human GGF2 (rhGGF2) to cultured NG2+ progenitors isolated from the contused adult rat spinal cord 3 days after SCI increased NG2+ cell numbers.

A therapeutic approach to improve functional recovery after central nervous system injury, like SCI, is to enhance the proliferation of these cells to yield more functional mature glia and improved myelination of surviving and regenerating axons. Provided herein are methods of treating spinal cord injury using GGF2 and compositions comprising GGF2. For example, provided is a method of treating spinal cord injury in a subject, comprising administering at least one dosage of less than 1 mg/kg of GGF2 to the subject. GGF2 and compositions comprising GGF2 may be referred to herein as therapeutic agents or agents.

Also provided herein are methods of promoting proliferation of neural stem cells (e.g., Sox 2 positive stem cells and comprising contacting the glial precursor cells with GGF2. For example, the contacting step is performed multiple times. The contacting steps can be performed daily for two, three, four, five, six, or seven days and or at least weekly for two, three, or four weeks. Optionally the contacting steps are performed in vitro or in vivo. Generally, the contacting step is performed in vivo within one day of central nervous system injury. Optionally, the method further comprises contacting the neural stem cells with a second agent, such as for example, FGF2. Optionally the second agent is not pituitary adenylate cyclase-activating peptide (PACAP) or prolactin. The in vitro method can be used to make cells for transplantation. Thus, provided herein is a method of treating a central nervous system injury in a subject by administering neural stem cells, glial precursor cells or progeny thereof to the subject, wherein the cells are made by the present method.

Also provided are methods of promoting revascularization of neural tissue following central nervous system injury in a subject comprising administering to the subject GGF2. The GGF2-mediated increase in pericytes improves revascularization after SCI to contribute to functional recovery. Optionally the administration steps if performed within one day of injury and optionally multiple doses are administered. For example, GGF2 is administered daily for two, three, four, five, six, or seven days and, optionally, GGF2 is administered weekly for two, three, or four weeks. The method can further comprise administering a second agent, such as for example, FGF2. Optionally the second agent is not pituitary adenylate cyclase-activating peptide (PACAP) or prolactin.

As used herein, GGF2 refers to the neural glial growth factor 2. Homologs, variants, and isoforms thereof having a proliferative effect can be used in the present methods. Nucleic acids that encode the GGF2 polypeptide sequences, variants, and fragments thereof are disclosed. These sequences include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequences.

As used herein, the term peptide, polypeptide or protein is used to mean a molecule comprised of two or more amino acids linked by a peptide bond. Protein, peptide, and polypeptide are also used herein interchangeably to refer to amino acid sequences. It should be recognized that the term polypeptide or protein is not used herein to suggest a particular size or number of amino acids comprising the molecule and that a polypeptide of the disclosure can contain up to several amino acid residues or more.

As with all peptides, polypeptides, and proteins, including fragments thereof, it is understood that additional modifications in the amino acid sequence of the variant GGF2 polypeptides can occur that do not alter the nature or function of the peptides, polypeptides, or proteins. Such modifications include conservative amino acids substitutions and are discussed in greater detail below.

The polypeptides described herein can be further modified and varied so long as the desired function is maintained. For example, a desired function is increase mylenation in the spinal cord and/or to provide functional improvement of one or more spinal cord injury symptom.

It is understood that one way to define any known modifications and derivatives or those that might arise, of the disclosed genes and proteins herein is through defining the modifications and derivatives in terms of identity to specific known sequences. Specifically disclosed are polypeptides which have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to GGF2 and variants provided herein. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math 2:482 (1981), by the identity alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by, for example, the algorithms disclosed in Zuker, Science 244:48-52 (1989); Jaeger et al, Proc. Natl. Acad. Sci. USA 86:7706-10 (1989); Jaeger et al, Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Protein modifications include amino acid sequence modifications. Modifications in amino acid sequence may arise naturally as allelic variations (e.g., due to genetic polymorphism), may arise due to environmental influence (e.g., by exposure to ultraviolet light), or may be produced by human intervention (e.g., by mutagenesis of cloned DNA sequences), such as induced point, deletion, insertion, and substitution mutants. These modifications can result in changes in the amino acid sequence, provide silent mutations, modify a restriction site, or provide other specific mutations. Amino acid sequence modifications typically fall into one or more of three classes: substitutional, insertional, or deletional modifications. Insertions include amino and/or terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at anyone site within the protein molecule Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional modifications are those in which at least one residue has been removed and a different residue inserted in its place. Such substitutions generally are made in accordance with the following Table 1 and are referred to as conservative substitutions.

TABLE 1
Amino Acid Substitutions

| Amino Acid | Substitutions (other are known in the art) |
|---|---|
| Ala | Ser |
| Arg | Lys |
| Asn | Gin |
| Asp | Glu |
| Cys | Ser |
| GIn | Asn |
| Glu | Asp |
| Gly | Pro, Ala |
| His | Asn, Gin |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

Modifications, including the specific amino acid substitutions, are made by known methods. By way of example, modifications are made by site specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis.

Provided herein are compositions containing the polypeptides, and nucleic acid molecules and a pharmaceutically acceptable carrier described herein. The herein provided compositions are suitable for administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize degradation of the active ingredient and to minimize adverse side effects in the subject.

Suitable carriers and their formulations are described in Remington: The Science and Practice of Pharmacy, 2^{nd} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the small molecule, polypeptide and/or nucleic acid molecule, to humans or other subjects.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatic ally, intracranially, nebulization/inhalation, intraspinally, subdurally or by installation via bronchoscopy. Optionally, the composition is administered by oral inhalation, nasal inhalation, or intranasal mucosal administration. Administration of the compositions by inhalant can be through the nose or mouth via delivery by spraying or droplet mechanism, for example, in the form of an aerosol. Administration is optionally into the central nervous system including into or on any dura layer and into the spinal cord.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, the nucleic acid molecule or polypeptide is administered by a vector comprising the nucleic acid molecule or a nucleic acid sequence encoding the polypeptide. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retorviruseso, Cold Spring Harbor Laboratory Press (1997), which is incorporated by reference herein for the vectors and methods of making them. The construction of replication-defective adenoviruses has been described (Berkner et al, J. Virol. 61:1213-20 (1987); Massie et al, Mol. Cell. Biol. 6:2872-83 (1986); Haj-Ahmad et al, J. Virol. 57:267-74 (1986); Davidson et al, J. Virol. 61:1226-39 (1987); Zhang et al, BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided polypeptides and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The provided polypeptides can be delivered by subviral dense bodies (DBs). DBs transport proteins into target cells by membrane fusion. Methods for making and using DBs are described in, for example, Pepperl-Klindworth et al, Gene Therapy 10:278-84 (2003).

The provided polypeptides can be delivered by tegument aggregates. Methods for making and using tegument aggregates are described in International Publication No. WO2006/110728.

Non-viral based delivery methods can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Palo Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV 40), adenovirus, retroviruses, hepatitis B virus, and most preferably cytomegalovirus (CMV), or from heterologous mammalian promoters, e.g. β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g. antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g., spinal cord injury). The term patient or subject includes human and veterinary subjects.

A subject at risk of developing a disease or disorder can be genetically predisposed to the disease or disorder, e.g., have a family history or have a mutation in a gene that causes the disease or disorder, or show early signs or symptoms of the disease or disorder. A subject currently with a disease or disorder has one or more than one symptom of the disease or disorder and may have been diagnosed with the disease or disorder. A subject with a spinal cord injury can include injury caused by any number of factors, including trauma or surgery. Demyelinating diseases include MS.

The methods and agents as described herein are useful for both prophylactic and therapeutic treatment. For prophylactic use, a therapeutically effective amount of the agents described herein are administered to a subject prior to onset (e.g., before obvious signs of spinal cord injury) or during early onset (e.g., upon initial signs and symptoms of spinal cord injury). Prophylactic administration can occur for several days to years prior to the manifestation of symptoms of spinal cord injury. Prophylactic administration can be used, for example, in the preventative treatment of subjects diagnosed with a genetic predisposition to spinal cord injury (e.g., in the case of spinal deformity or achondroplasia) or after spinal cord injury. Therapeutic treatment involves administering to a subject a therapeutically effective amount of the agents described herein after diagnosis or development of spinal cord injury.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Optionally, a dosage of less than 1 mg/kg GGF2 or a composition comprising 1 mg/kg GGF2 is administered to the patient. A dosage of less than 1 mg/kg includes 0.9, 0.8, 0.7, 0.6, 0.5, 0.4, 0.3, 0.2, 0.1 or any amount between 1 and 0 mg/kg. This dosage can be administered once, or repeated one or more times over a period of days, weeks or years. Thus, the total dosage may be greater than 1 mg/kg. Optionally, the dosage administered one or more times is 0.8 mg/kg. Optionally, the dosage is given at least 24 hours following the spinal cord injury. If a series of dosages is administered over time, then the first dosage is optionally administered at least 24 hours after the spinal cord injury. Each additional dosage can be administered at some temporal duration subsequent to the first administered dosage. For example, a dosage of less than 1 mg/kg can be administered to a subject each day for two or more days, wherein the days are optionally concurrent or optionally not concurrent. If the days are not concurrent then the second dosage may follow the first by any number of days. A dosage of less than 1 mg/kg can be followed with a dosage of 1 mg/kg or higher.

Optionally, the GGF2 or composition thereof is administered in combination with other agents, including, for example, anti-inflammatory agents including steroidal and non-steroidal anti-inflammatory agents. Optionally, the steroid is prednisone.

Optionally, the GGF2 is administered in conjunction with surgery, for example, in the case of spinal cord injury, to stabilize a vertebral fracture. GGF2 can also be used prophylactically with any spinal surgery when injury or inflammation of the spinal cord is a possibility, including for example, laminectomy, spinal fusions, or the like.

A dosage of less than 1 mg/kg offers advantages over a dosage of 1 mg/kg, including, for example, a reduced risk of side effects and a reduced cost and/or the optimization of a dose and/or dosing regimen based upon the identification of a V-shaped dose-response curve.

One of skill in the art selects the dosage and mode of administration based on a number of factors including the severity of the disease or injury or the risk of disease or injury, the age and weight of the subject, and the like.

As used herein the terms treatment, treat, or treating refers to a method of reducing the effects of a disease or condition (e.g., spinal cord injury) or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. The effect of the administration to the subject can have the effect of, but is not limited to, reducing the symptoms of the condition, a reduction in the severity of the condition, or the complete ablation of the condition.

As used herein, the terms prevent, preventing, and prevention of a disease or disorder refers to an action, for example, administration of a therapeutic agent, that occurs before or at about the same time a subject begins to show one or more symptoms of the disease or disorder (e.g., spinal cord injury), which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1

Demyelination and abnormal remyelination of axons are major pathological consequences of chronic spinal cord injury (SCI). Axons lacking proper myelination are unable to efficiently conduct action potentials. Restoration of proper conduction could potentially lead to functional improvements below the injury site. The adult spinal cord contains a pool of endogenous glial precursor cells which spontaneously respond to SCI with increased proliferation. A therapeutic approach to improve functional recovery after SCI is to enhance the proliferation of these cells to yield more functional mature glia and improved myelination of surviving and regenerating axons. Basic fibroblast growth factor (FGF2) and glial growth factor 2 (GGF2), are two mitogens found to be upregulated after SCI, enhance oligodendrogenesis in both in vitro and in vivo model systems. These factors can be used to enhance long-term functional recovery in vivo in a mouse model of SCI.

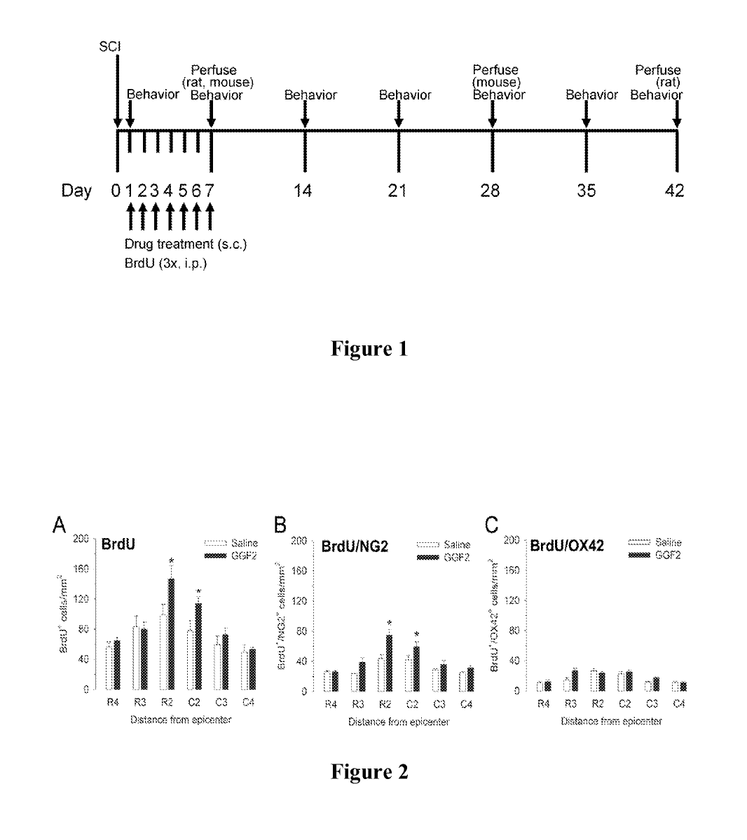

As outlined in FIG. 1, adult mice were subjected to a standardized incomplete contusive spinal cord injury at the ninth thoracic vertebra (T9) using the Infinite Horizons injury device (60 kdyn force). Injured mice (n=11 per group) were injected once daily, for 7 days, beginning one day post injury (dpi) with FGF2 (0.02 mg/kg, sc), GGF2 (0.8 mg/kg, sc), a combination of FGF2+GGF2, or with saline alone.

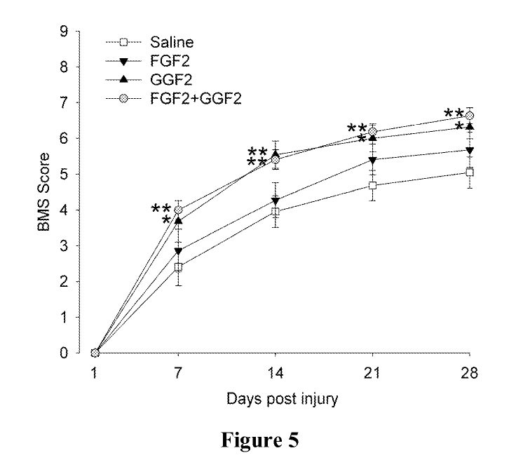

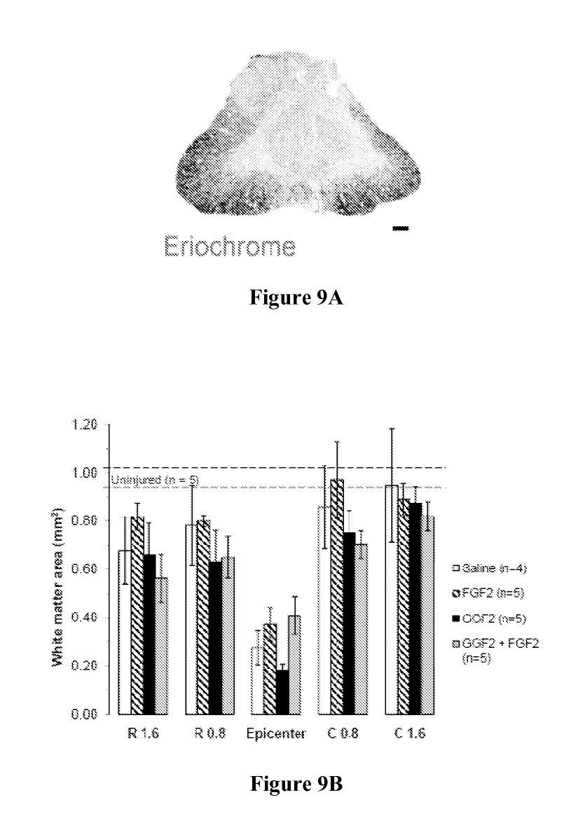

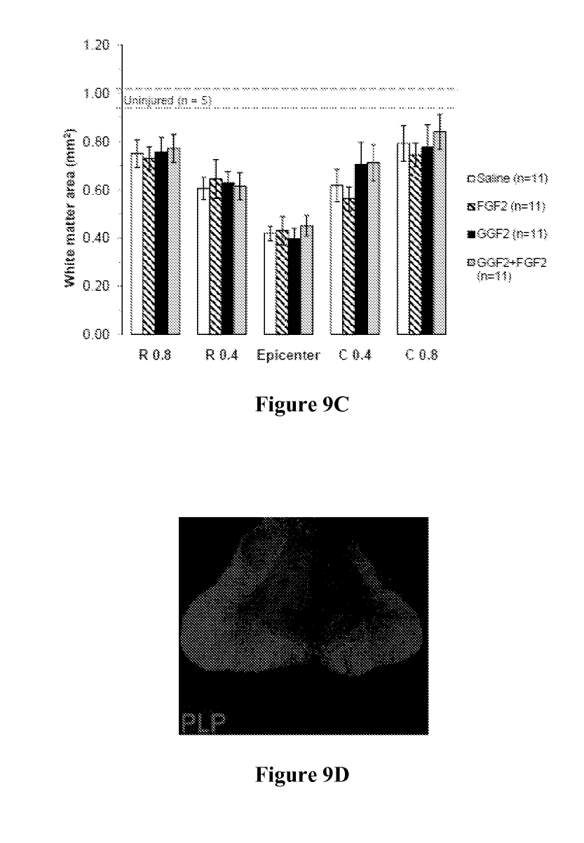

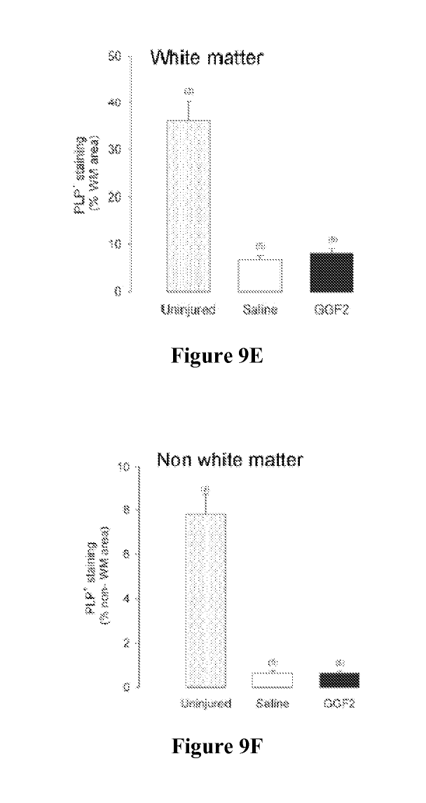

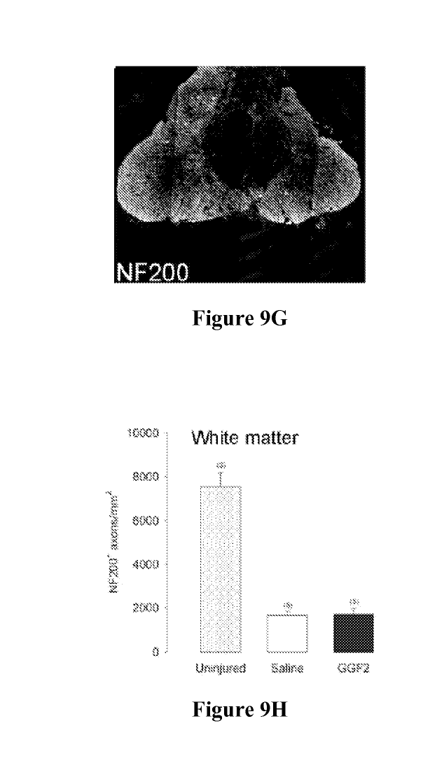

Hindlimb functional recovery was assessed using the Basso Mouse Scale (BMS) open field locomotor score at 1, 7, 14, 21, and 28 days post injury. As shown in FIG. 5, treatment with FGF2+GGF2 or GGF2 alone resulted in a significant improvement in BMS score compared to saline treated controls. At 28 days post injury, spinal cords were obtained for histological assessment. There was no significant effect of growth factor treatment on spared white matter area as measured by eriochrome-cyanine staining (FIG. 9C).

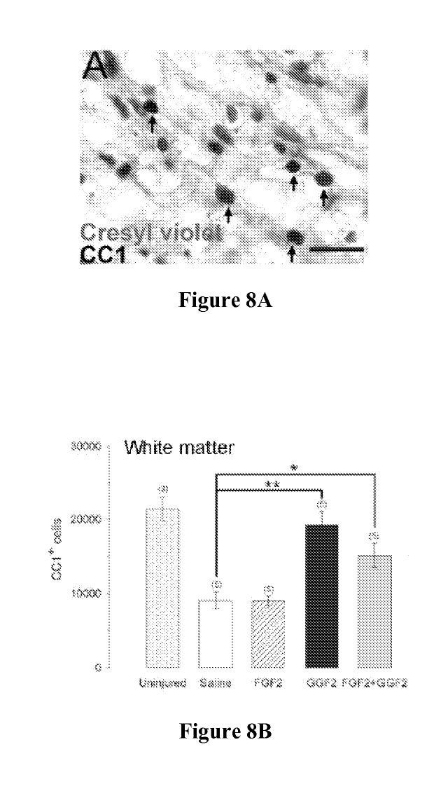

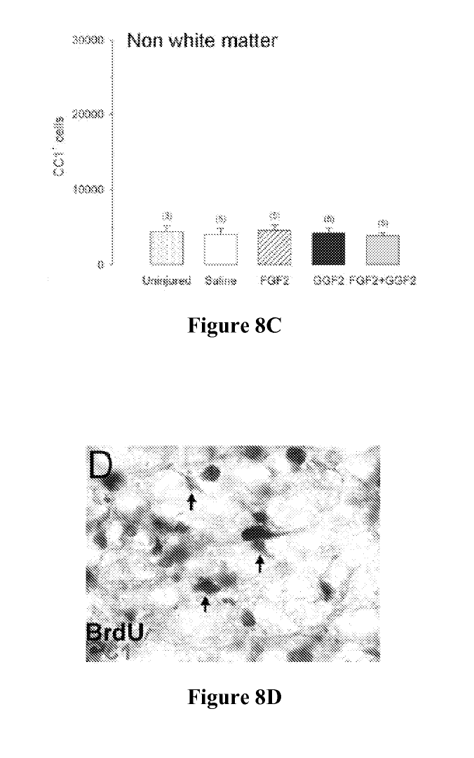

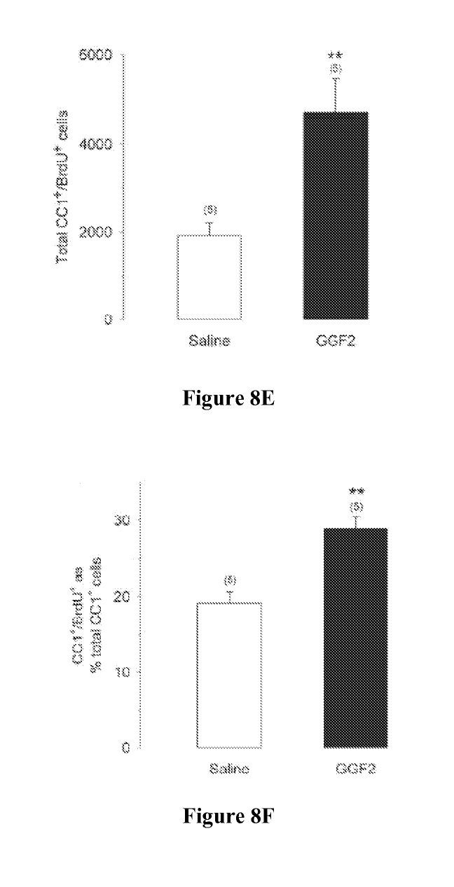

For further analysis, representative subsets of spinal cords were selected from saline treated (n=5) and GGF2 treated (n=5) mice. Estimates of axon number using NF200 staining showed no difference between treatment groups (FIG. 9I). However, injury epicenters of mice that received GGF2 or FGF2+GGF2 treatment exhibited a significant increase in number of mature oligodendrocytes (FIG. 8B) as measured by stereological assessment of CC1 immunostaining. Thus, delayed treatment (1 day post injury) of incomplete contusive SCI with FGF2+GGF2 increases replacement of lost oligodendrocytes and improves long term functional recovery from spinal cord injury, supporting the enhancement of endogenous recovery mechanisms as a therapeutic strategy for SCI. More specifically, treatment with GGF2 increases the total number of oligodendrocytes chronically after SCI in the ventral lateral white matter, a region of interest (ROI) that contains important descending pathways for control of hind limb motor function. In addition to increasing proliferation of oligodendrocytes, by 7 days after SCI, treatment with GGF2 significantly increases other NG2-expressing cells such as PDGF receptor beta-expressing pericytes associated with blood vessels. Furthermore, treatment with GGF2 increases the number of Sox-2 positive neural stem cells.

### Example 2

Treatment with GGF2 alone at a dose of 0.8 mg/kg was effective in significantly improving recovery after SCI. As shown in FIG. 5, open field locomotion was significantly improved from 1 week through 4 weeks after injury. GGF2 alone was as effective as the combination of FGF2+GGF2. As shown in FIG. 9C, treatment with GGF2 did not significantly affect the area of spared white matter at 4 weeks after injury. However, the total number of mature oligodendendrocytes in the white matter was significantly increased to more than twice the number in the vehicle (saline) treated control group (FIG. 8B). As previous studies have shown that about half the initial mature oligodendrocytes are lost in spared white matter within the first 24 hours after SCI, the results indicate that treatment with 0.8 mg/kg GGF2 alone, beginning at 24 hours after SCI is highly effective in stimulating the production of replacement oligodendrocytes. These cells are important in myelinating axons, as shown in FIG. 8B, thus improve axonal function and enhance recovery of function from SCI.

### Materials and Methods

Spinal Cord Injury (SCI).

Surgery was performed on female Sprague Dawley rats (Zivic Miller Laboratories, Inc.; Pittsburgh, Pa.) weighing 225-300 g. Rats were anesthetized with chloral hydrate (360 mg/kg intraperitoneally (i.p.)), and a laminectomy was performed at the level of thoracic vertebra 8 (T8) to expose a circle of dura. Contusion was produced by dropping a 10 g weight from a height of 2.5 cm onto an impounder positioned on the exposed dura (Wrathall et al., Exp. Neurol. 88:108-22 (1985)). Adult CNP-EGFP (Yuan et al., J. Neurosci. Res. 70:529-45 (2002)) male and female mice (15-20 g), in which all cells of the oligodendrocyte lineage express enhanced green fluorescent protein, were anesthetized with avertin (2,2,2-tribromoethanol, 0.4-0.6 mg/g), and a laminectomy was performed at T9 to remove the part of the vertebra overlying the spinal cord, exposing a circle of dura. The spinal column was stabilized via the lateral processes using transverse clamps at T7 and T10. A moderate contusion injury was produced using the Infinite Horizon (Precision Systems & Instrumentation; Fairfax Station, Va.) spinal cord impactor with a force of 60 kdyn (Nishi et al., J. Neurotrama 24:674-89 (2007)). After SCI, rats and mice were kept on highly absorbent bedding and their bladders manually expressed twice daily until a reflex bladder was established (7-14 days after SCI). Rats and mice were tested behaviorally to confirm injury at 24 hours after contusion. Animals were subsequently assigned to treatment groups according to a randomized block experimental design.

Drug Treatments.

Recombinant human glial growth factor 2 (GGF2) was provided by Acorda Therapeutics (Hawthorne, N.Y.) and fibroblast growth factor 2 (FGF2) was from Peprotech (Rocky Hill, N.J.). Drugs were dissolved in sterile saline and administered subcutaneously, once daily from day 1 through day 7 after SCI, as shown in FIG. 1. Rats received 1 mg/kg GGF2, while mice received FGF2 (0.02 mg/kg), GGF2 (0.8 mg/kg), or FGF2 (0.02 mg/kg)+GGF2 (0.8 mg/kg). Vehicle controls received equivalent volumes of saline.

Behavioral Testing.

Rat hind limb locomotor recovery was assessed at 1 d post injury and weekly thereafter for up to 6 weeks using the Basso, Beattie and Bresnahan (BBB) open field expanded locomotor score (Basso et al., J. Neurotrauma 12:1-21 (1995)). The test is a rating scale of 0-21, where animals with complete hind limb paralysis are scored 0, and animals with normal locomotion are scored 21. Rats were also scored on a battery of tests to determine recovery of hind limb motor and sensory function including: open field locomotion (motor score); withdrawal reflex to hind limb extension, pain, and pressure; foot placing, toe spread, and righting reflexes; maintenance of position on an inclined plane, and swimming tests. Results of these tests are reported as a Combined Behavioral Score [CBS (Gale et al., Exp. Neuro. 88:123-34 (1985))]. Rats with complete paralysis that are abnormal on all tests score 100 on the CBS while rats with normal function receive a score of 0. All rats were tested without knowledge of treatment group.

Mice were tested for hind limb function in open field locomotion on day 1 post injury and weekly thereafter for up to 4 weeks using the Basso mouse scale (BMS) for locomotion (Basso et al., Exp. Neurology 139:244-56 (1996)). This scale ranges from 0-9 with a score of 0 representing no movement of the hind limbs and a score of 9 representing normal use in coordinated, weight-bearing locomotion. Behavioral testing was performed by investigators who were blind to the treatment groups until all primary data was collected and analyzed.

Perfusion and Preparation of Tissue for Histopathology.

At the specified times after injury, subjects were anesthetized and transcardially perfused with phosphate buffered saline followed by 4% buffered paraformaldehyde (PFA). Spinal cords were removed and postfixed overnight in PFA, and cryopreserved in a sucrose gradient (1 h in 10% sucrose, 1 h in 15% sucrose, 1 h in 20% sucrose, and overnight in 30% sucrose). Segments of spinal cord centered on the injury epicenter were removed and embedded in OCT compound (Tissue-Tek®; Sakura Finetek USA, Inc.; Torrance, Calif.), and stored at −20° C. Serial 20 μm coronal sections were cut and slides were stored at −20° C. Representative slides were stained with eriochrome-cyanine to label myelin (Grossman et al., Exp. Neurology 168:273-82 (2001)) in order to assess tissue morphology and determine injury epicenter locations in each spinal cord.

White Matter Sparing.

Residual white matter area was calculated at the injury epicenter as well as at points rostral and caudal to the epicenter by quantifying the total area stained by eriochrome-cyanine. Images were taken at 2.5× magnification and analyzed using NIH ImageJ software. The threshold level of each 8-bit image was set to display only eriochrome positive pixels, and total eriochrome-positive area was calculated for each section.

Immunohistochemistry.

Immunohistochemistry was performed on spinal cord sections at specified distances rostral and caudal to the injury epicenter. Slides were allowed to equilibrate to room temperature for 1 hour, then incubated with 10% buffered formalin for 10 minutes. Sections were washed with PBS and incubated with 0.3% H₂O₂ for 30 minutes to quench endogenous peroxidase activity. For staining of myelin proteins P0 and proteolipid protein (PLP), slides were subjected to an alcohol gradient to remove lipids from sections. Slides were then rinsed with PBS and blocked for 1 hour with 10% serum in PBS+0.3% Triton X-100. Sections were then incubated overnight at 4° C. with primary antibodies (Table 2). Primary antibodies against APC [CC1] (Abcam; Cambridge, Mass.), OX42 (Serotec), and BrdU (BD Biosciences; Franklin Lakes, N.J.) were monoclonals raised in mouse. Primary antibodies against NG2 (Millipore; Billerica, Mass.), Sox2 (Millipore), and NF200 (Sigma; St. Louis, Mo.) were polyclonals raised in rabbit. Primary antibodies against P0 and PLP were polyclonals raised in chicken (Ayes Labs, Inc.; Tigard, Oreg.). Slides were washed with PBS and incubated with secondary antibody for 1 hour at room temperature. Fluorescent immunohistochemistry was performed using Cy3-conjugated and/or Cy5-conjugated goat secondary antibodies directed against mouse, rabbit, or chicken immunoglobulins (Jackson Immunoresearch; West Grove, Pa.) diluted in PBS plus 1% serum and 0.3% Triton X-100. Finally, slides were washed and mounted with Vectashield containing DAPI (Vector Laboratories; Burlingame, Calif.) Immunoperoxidase staining was carried out by incubating slides with biotinylated goat anti mouse secondary antibody (Vector Laboratories) followed by avidin-biotin peroxidase complex (ABC Elite, Vector Laboratories). Slides were washed, and 3,3′ diaminobenzidine (DAB) with nickel enhancement (Vector Laboratories) was then applied, yielding a black reaction product. Slides were subsequently dehydrated and mounted with Permount. All immunohistological staining experiments were carried out with appropriate positive control tissue as well as secondary-only negative controls.

TABLE 2
Primary Antibodies Used

| Antibody | Species | Manufacturer | Catalog no. |
|---|---|---|---|
| APC [CC1] | Mouse | Abcam | Ab 16794 |
| BrdU | Mouse | Becton Dickinson | 347580 |
| CD11b [OX42] | Mouse | Abd Serotec | MCA275R |
| Sox2 | Rabbit | Millipore | AB5603 |
| Neurofilament 200 | Rabbit | Sigma | N4142 |
| NG2 | Rabbit | Millipore | AB5320 |
| P0 | Chicken | Aves | PZO |
| PLP | Chicken | Aves | PLP |

Cell Proliferation Studies.

Rats and mice received intraperitoneal injections of bromodeoxyuridine (BrdU, 17 mg/kg) at multiple time points (as specified in Results) to label cells in S phase of mitosis during the first week after injury. To detect BrdU in spinal cord, sections were treated with 10% formalin and washed with PBS followed by treatment with 2M HCl for 25 minutes at 37° C. Tissue was then neutralized using 0.1M borate buffer (pH 8.5) for 10 minutes. The sections were washed with PBS and endogenous peroxidases were quenched with 0.3% H₂O₂, followed by a 1 hour blocking step in 20% serum. Tissue was then incubated with mouse anti-BrdU antibody (BD Biosciences) for 1 hour at room temperature. BrdU positive cells were detected using biotinylated goat anti-mouse secondary followed by avidin-biotin peroxidase complex and subsequent DAB staining as described above.

To identify mature oligodendrocytes that arose in the first week after injury (during the time of BrdU exposure), in tissue chronically after SCI, BrdU stained sections were washed, and blocked for 30 minutes in 20% serum. Slides were then incubated with mouse anti APC[CC1] (Abcam) for 1 hour at room temperature. Biotinylated goat anti-mouse secondary antibody (Vector Laboratories) was applied, followed by Vector ABC Elite and Vector NovaRed chromogen to yield a red/brown reaction product.

Unbiased Stereology.

CC1⁺ Cells:

5 representative subjects (based on BMS score) from the 28 day mouse study were selected from each treatment group for quantification of mature oligodendrocytes (CC1⁺ cells) using unbiased stereology. 3 sections per animal, spaced 200 μm apart and centered on the injury site, were included for counting using the optical fractionator method with the aid of Stereo Investigator software (MBF Bioscience; Williston, Vt.). Sections were stained using the immunoperoxidase method with Ms×APC(CC1) as the primary antibody and DAB as the chromogen. Sections were counterstained with cresyl violet to visualize nuclei. Contours outlining spared white matter and non-white matter were traced onto each section based on the eriochrome staining of adjacent sections. A sampling grid comprised of 180 μm×180 μm squares was laid over each section. Cells were counted at 100× in a 45 μm×45 μm counting frame within each square of the counting grid. These parameters were established to allow for CE values of CC1⁺ cell counts to be <0.10. CC1⁺ mature oligodendrocytes were counted throughout the spared WM and non-WM in each section.

CC1⁺/BrdU⁺ Cells:

Sections adjacent to those used for the CC1⁺ cell counting study were used for the CC1⁺/BrdU⁺ cell counting study. 3 sections per animal, spaced 200 μm apart and centered on the injury site, were included for counting using the optical fractionator method with the aid of Stereo Investigator software. Sections were stained as described above. A sampling grid comprised of 140 μm×140 μm squares was laid over each section. Cells were counted at 100× in a 60 μm×60 μm counting frame within each square of the counting grid. Double labeled CC1⁺/BrdU⁺ cells were counted only if at least ¾ of the black BrdU⁺ nucleus was surrounded by the red/brown stain representing CC1.

Region of Interest Counting (Rats).

As in previous studies (Grossman et al., Exp. Neurology 168:273-82 (2001); Rosenberg and Wrathall, J. Neurosci. Res. 66:191-202 (2001); Zai and Wrathall, Glia 50:247-57 (2005)), cells were counted within a reticule of specified area (0.0625 mm²) positioned in the ventromedial region of the residual white matter at defined locations rostral and caudal to the injury epicenter. The cell counts for each rat are averages of bilateral counts on each of three spinal cord sections (6 samples) at each distance.

Region of Interest Counting (Mice).

Images were captured at 60× using an Olympus FV300 laser scanning confocal microscope (Olympus; Center Valley, Pa.). Cells were counted within a region of interest (ROI) of 0.02 mm² in the left and right ventrolateral white matter between the lesion border and the outer perimeter of the spinal cord tissue. Ventral and ventrolateral areas of the spinal cord are involved in hindlimb function and sparing of these areas contributes greatly to functional recovery. This area was found to be free of overt lesion in all subjects. Cells were counted at the injury epicenter and sections 200 μm rostral and caudal to the epicenter. The mean cells/mm² value for each subject is therefore determined from cell counts at 6 separate regions of interest.

PLP Quantification.

20× tilescan (4×4) images of PLP labeled sections were obtained using a Zeiss LSM 510 laser scanning confocal microscope. 3 sections per animal, spaced 200 μm apart and centered on the injury site, were included for quantitation. Images were opened in NIH ImageJ 1.44 m, and the scale set to reflect the pixel/μm ratio of the original image. The total area of each section was determined by outlining the section with the polygon selection tool. Images were converted to 8-bit format and the threshold set to reflect PLP staining. PLP staining was expressed as the percentage of the total area of the spinal cord. Next, the lesion area was outlined using the polygon selection tool based on the observed PLP staining in each section. The lesion edge was defined as the line separating residual WM (extensive PLP staining) from non-WM areas that showed sparse myelin staining. The area outside of the traced non-WM area was then removed, and PLP percentage area within the lesion was determined. PLP staining in spared WM was determined by subtracting the non-WM PLP staining from the total PLP staining.

NF200 Quantification.

20× tilescan (4×4) images of NF200 and PLP double labeled sections were obtained using a Zeiss LSM 510 laser scanning confocal microscope (Carl Zeiss, Inc.; Thornwood, N.Y.). 3 sections per animal, spaced 200 μm apart and centered on the injury site, were included for quantitation. Images were opened separately in NIH ImageJ 1.44 and stacked. The lesion area was outlined based on the observed PLP staining in each section. The lesion edge was defined as the line separating spared WM (extensive PLP staining) from non-WM areas that showed sparse myelin staining. Parameters for particle size and circularity were set to include only NF200⁺ axons. Total NF200⁺ particles in spared white matter as well as in non-white matter were quantified at the injury epicenter and at points 200 μm rostral and caudal.

P0 Quantification.

20× tilescan (4×4) images of P0 labeled sections were obtained using a Zeiss LSM 510 laser scanning confocal microscope. 3 sections per animal, spaced 200 μm apart and centered on the injury site, were included for quantitation. P0 staining was expressed as the percentage of the total area of spinal cord as described for PLP quantitation. The lesion area was outlined using the polygon selection tool based on the observed PLP staining in the adjacent section. P0 percentage area within the lesion and in residual WM was determined as described for PLP staining.

Sox2 Cell Counting.

20× tilescan (4×4) images of Sox2 labeled sections from CNP-EGFP mice were obtained using a Zeiss LSM 510 laser scanning confocal microscope. 3 sections per animal, spaced 200 μm apart and centered on the injury site, were included for quantitation. For total Sox2 cell counts, the Cy3 channel of each image was converted to an 8-bit image in NIH ImageJ 1.44. The total area of each section was determined by outlining the section with the polygon selection tool. The threshold was set to reflect the staining in the original image. Size and circularity limits were set to include only Sox2 stained cells. For Sox2/CNP-EGFP⁺ cell counts, the Cy3 and EGFP channels of each image were opened separately in Adobe Photoshop 7.0 (Adobe; San Jose, Calif.) and merged. Zoom was set to 286% and a 360×360 pixel grid was laid over each image. Double labeled cells were counted manually in each square of the grid. Cells that appeared to be double labeled were confirmed by sequentially turning off each image layer.

Sampling and Statistical Analysis.

In all cases, the number of subjects served as the sample size. Data are reported as mean±SEM. Significance was generally determined using repeated measures ANOVA, with time after injury (behavior), or location of the section with respect to the epicenter (cell counting) as the repeated measure of treatment effect. If an overall significant effect of treatment was detected, Tukey's post-hoc test was used to assess when/where the differences were significant. Student's t-test was also used where appropriate. Significance was set at p<0.05.

### Results

To determine if systemic GGF2 treatment during the first week after spinal cord injury (SCI) could increase proliferation of endogenous precursor cells, SCI rats were given GGF2 (1 mg/kg s.c., n=8) or saline (s.c., n=8) once daily for 1 week, beginning 24 hours after injury. Animals were injected with bromodeoxyuridine (BrdU, 17 mg/kg, i.p.) on days 2, 3, and 4 after injury to label cells in the S-phase of mitosis. As shown in FIG. 2A, GGF2 treatment significantly increased the number of BrdU-labeled cells by 7 days after SCI in residual ventromedial white matter (VMWM) at locations both 2 mm rostral and 2 mm caudal to the epicenter. GGF2 treatment also significantly increased the number of BrdU-labeled NG2⁺ cells in these same locations (FIG. 2B). In all locations studied, approximately 50% of the total proliferating cells were NG2⁺. Macrophages and monocytes infiltrate the injury site in the week following injury in the rat model of contusive SCI. To study whether there was an effect of GGF2 treatment on these cells, spinal cord sections were immunolabeled with anti-BrdU antibody and a monoclonal antibody against the microglia/macrophage marker OX42. No significant difference was found in OX42⁺/BrdU⁺ cell number between saline and GGF2 treated groups at any of the locations examined (FIG. 2C).

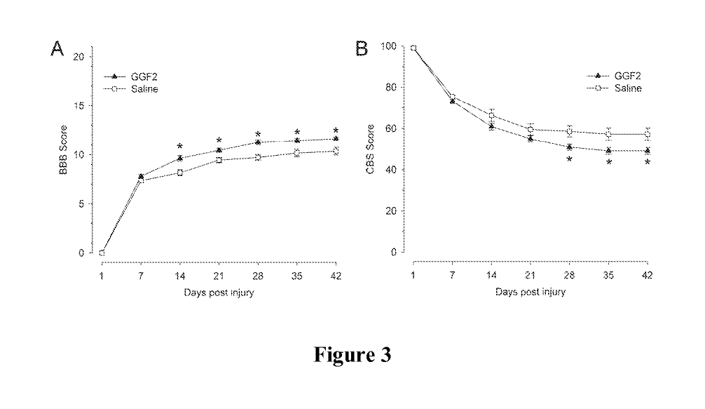

As 1 week of GGF2 treatment increased proliferation of NG2⁺ cells near the injury site, this same treatment strategy was examined to determine whether long-term functional recovery after SCI was influenced. The behavior of GGF2 (n=11) and saline (n=11) treated rats was assessed over the course of 6 weeks after injury. Testing was performed 1 day after injury and weekly thereafter using the BBB test of hind limb locomotor function and the Combined Behavioral Score (CBS), an evaluation of overall hind limb sensory-motor deficits. Both tests showed a significant beneficial effect of GGF2 treatment on functional recovery (FIGS. 3A and 3B). The effect of treatment was statistically significant beginning at week 2 as measured by BBB score (FIG. 3A) and beginning at week 4 as measured by CBS score (FIG. 3B). Both measures showed significantly improved recovery chronically at 4-6 weeks after SCI.

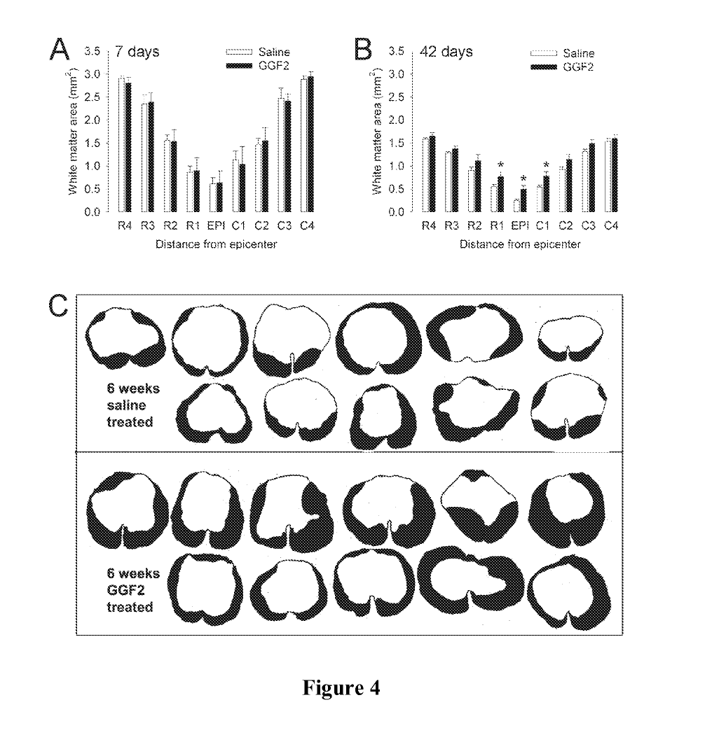

To determine if the observed behavioral improvement could be attributed to increased remyelination of spared axons, spinal cord sections from SCI rats perfused at 7 days and 42 days post injury were stained with eriochrome-cyanine and WM area was quantified at the injury epicenter and at 1 mm intervals 1-4 mm rostral and caudal to it (FIG. 4). No difference in WM area was detected between treatment groups at 7 days post injury (FIG. 4A). However, GGF2 treatment resulted in a significant increase in WM area at 42 days post injury at the injury epicenter and at locations 1 mm rostral and 1 mm caudal to the epicenter (FIG. 4B). Tracings of eriochrome stained profiles at the injury epicenters of each subject illustrate the differences in myelinated white matter area between saline and GGF2 treated subjects (FIG. 4C).

Like GGF2, FGF2 is an endogenous glial mitogen whose expression is up-regulated near the site of injury in the rat spinal cord in the first week after SCI. Application of a combination of FGF2+GGF2 to cultures of NG2⁺ cells isolated from injured rat spinal cord (3 days post injury) stimulated greater proliferation than either factor alone. Furthermore, 1 week treatment with systemic FGF2+GGF2 following SCI in mice increased total NG2⁺ cells as well as mature oligodendrocytes in residual WM at the epicenter at 8 days post injury. The hypothesis that a combination of FGF2 and GGF2 could provide an additive or even synergistic beneficial effect on functional recovery after SCI was sought to be determined. To permit a more detailed study of the role of oligodendrocytes in improved recovery CNP-EGFP mice were used. CNP-EGFP mice, in which all cells of the oligodendrocyte lineage express EGFP, were subjected to standardized contusive SCI.

In the short-term mouse study, four groups of CNP-EGFP mice (n=5/group) were subjected to incomplete SCI and assigned to receive either saline, FGF2 (0.02 mg/kg), GGF2 (0.8 mg/kg), or FGF2 (0.02 mg/kg)+GGF2 (0.8 mg/kg) treatment. Drug treatments were administered beginning at 24 hours after injury and once daily for 7 days, as in the rat studies. Mice were also injected with bromodeoxyuridine (BrdU, 17 mg/kg, i.p.) on days 2, 4, and 7 after injury to label cells in the S-phase of mitosis. Hind limb locomotor recovery was assessed at 1 day and 7 days post injury using the Basso Mouse Scale (BMS) for locomotion. The treatment paradigm for the long-term mouse study was organized in the same way, however the number of subjects was 11/group, and the behavioral studies were extended out to 28 days post injury.

None of the drug treatments influenced the functional recovery of SCI mice at 7 days post injury. However, both GGF2 and FGF2+GGF2 treatments significantly improved long-term functional recovery compared to saline treated subjects (FIG. 5). At 28 days post injury, treated subjects displayed greater locomotor coordination and more consistent plantar stepping with their hind paws as compared with controls. FGF2 treatment had no significant effect on functional recovery, suggesting that GGF2 alone is sufficient to evoke functional improvements after SCI.

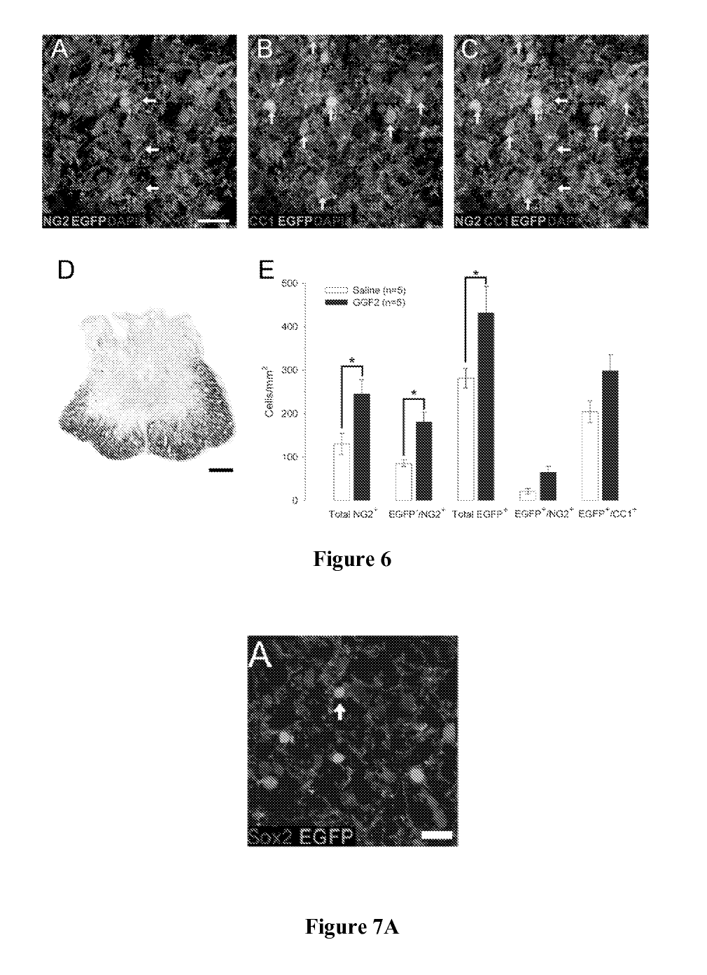

Tissue from subjects perfused at 7 days post injury was used to examine the effect of GGF2 treatment on the number of several different cell types including NG2⁺ cells (FIGS. 6A and 6E), oligodendrocyte lineage cells (FIGS. 6B and 6E), and mature oligodendrocytes (FIGS. 6C and 6E) in residual WM. By 1 week after injury, GGF2 treatment significantly increased the total number of NG2⁺ cells as well as the non-oligodendrocyte lineage (EGFP⁻/NG2⁺) NG2 cell population in the ventro-lateral WM (VLWM) at the injury epicenter compared to saline treated controls (FIG. 6D). The total number of oligodendrocyte lineage cells (EGFP⁺, FIG. 6D) in this region was also increased in GGF2 treated subjects. GGF2 treatment showed a tendency toward an increased number of mature oligodendrocytes (FIG. 6D, p=0.06 vs. saline) although the effect size of treatment did not reach statistical significance.

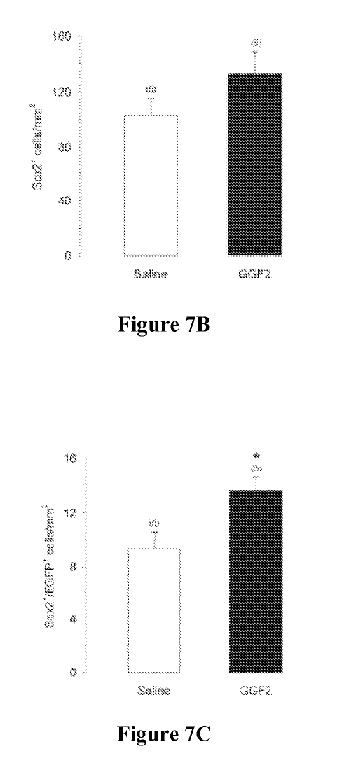

Sox2 is a transcription factor expressed in neural stem cells during development of the CNS (Collignon et al., Development 122:509-20 (1996)) and acts to regulate stem cell self-renewal and pluripotent properties (Fong et al., Stem Cells 26:1931-8 (2008); Kim et al., Nature 454:646-50 (2008)). Spinal cord expression of Sox2 is largely limited to ependymal cells lining the central canal in uninjured mice. However, Sox2 expression is significantly increased in residual white matter after SCI and is maximal at 7 days after injury. Quantification of CNP-EGFP⁺ cells immuno-labeled with Sox2 antibody revealed that GGF2 treatment significantly increased the number of oligodendrocyte lineage cells expressing Sox2 at the injury epicenter at 7 days after injury (FIG. 7C).

Cells dividing in the first week after SCI survive and differentiate into mature oligodendrocytes both in vitro and in vivo. Whether growth factor treatment could enhance the number of cells that differentiate into mature oligodendrocytes chronically after SCI was examined (FIG. 8). Sections from a subset of 5 of the 11 mice from each treatment group were used for cell counting using the optical fractionator method of unbiased stereology (Stereoinvestigator, MBF Biosciences; Williston, Vt.). The subjects chosen were representative of their entire respective treatment groups based on BMS score. Total mature oligodendrocytes (CC1⁺ cells) were counted in WM and non-WM in a 400 μm length of spinal cord centered on the injury site. Both FGF2+GGF2 and GGF2 alone treatments significantly increased the number of mature oligodendrocytes in white matter compared to saline treated controls (FIG. 8B), while no effect of any drug treatment was seen in lesion/non-WM areas (FIG. 8C). GGF2 treatment resulted in a nearly 2-fold increase in the number of mature oligodendrocytes in WM relative to saline controls. Double-immunolabeling experiments for BrdU and CC1 (FIG. 8D) were used to detect oligodendrocytes derived from cells dividing during drug treatment. GGF2 treatment led to an approximately 2.5-fold increase in CC1⁺/BrdU⁺ cells in WM (FIG. 8E). The percentage of total mature oligodendrocytes at 28 days post injury derived from cells that were proliferating in the first week after SCI (FIG. 8F) was also significantly increased by GGF2 treatment.

To assess whether the increased oligodendrocyte number after GGF2 treatment resulted in greater myelination of axons in the injured spinal cord, total white matter area in 28 day post injury spinal cord sections was quantified over a 1.6 mm segment of spinal cord centered on the injury epicenter using eriochrome-cyanine staining (FIG. 9A). No differences in WM area between treatment groups were observed at any of the locations tested (FIG. 9B). GGF2 also had no detectable overall effect on CNS-specific myelination of the injury epicenter as quantified by assessment of PLP⁺ staining area (FIGS. 9C-9E). No effect on axonal sparing with GGF2 treatment was detected, as both control and treated subjects showed similar NF200 staining (FIGS. 9F-9H).

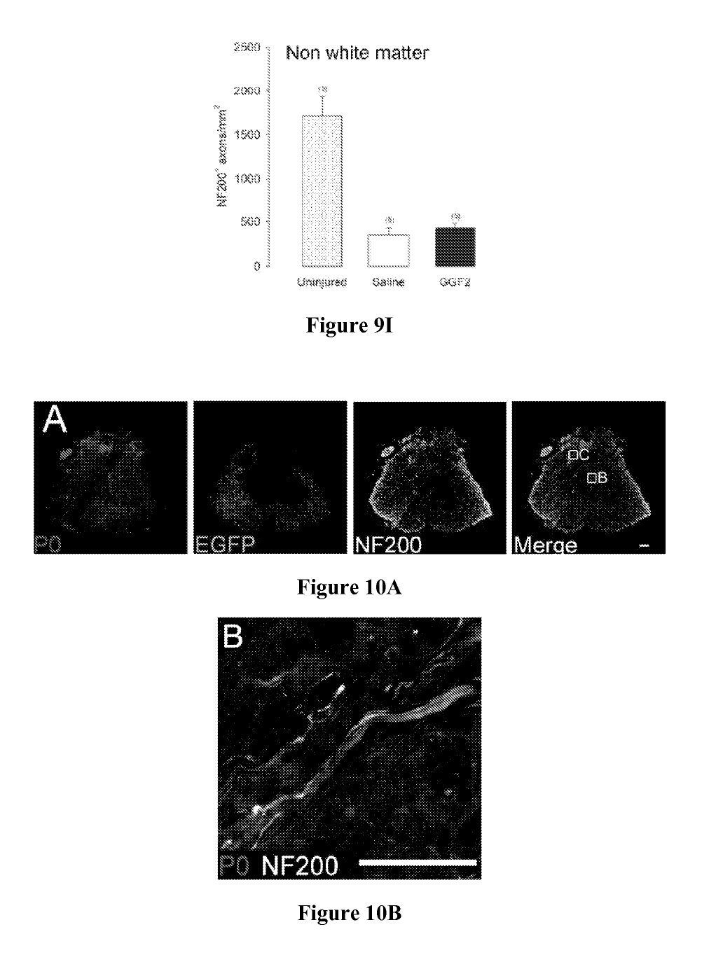

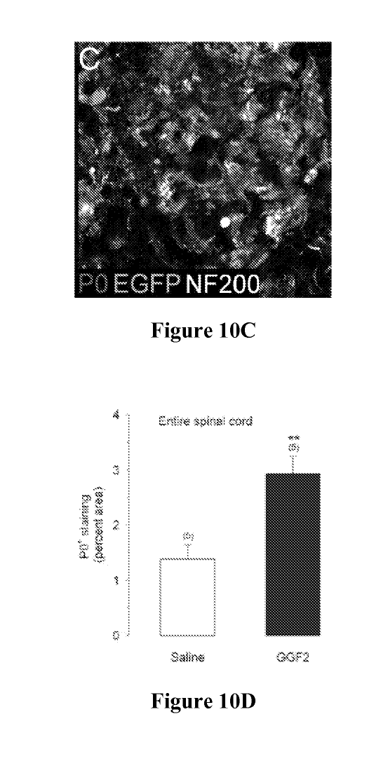

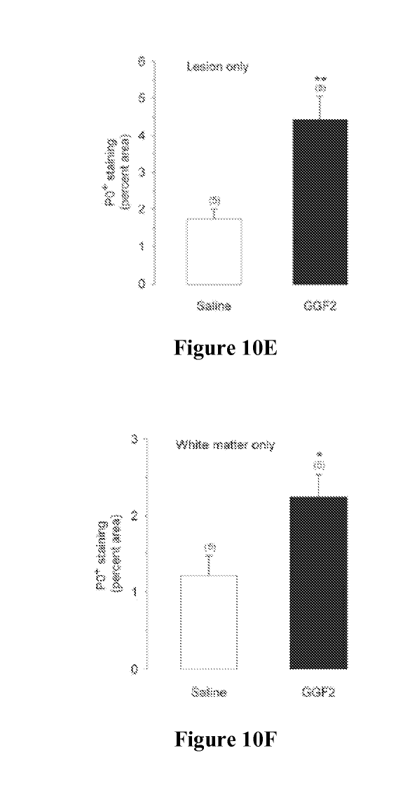

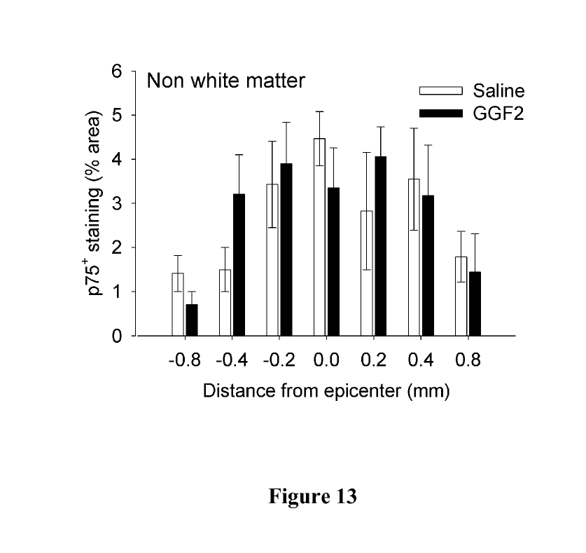

Schwann cells infiltrate the injury site after SCI, and likely contribute to mechanisms underlying spontaneous functional improvement. To assess whether GGF2 treatment influenced PNS-specific myelination of the injury site chronically, sections from the injury epicenter ±200 μm at 28 days after injury were stained for the peripheral myelin structural protein P0 (FIG. 10). GGF2 treated subjects had approximately twice the amount of P0 staining observed in saline treated controls (FIG. 10D). GGF2 treatment led to increased P0 staining both in the lesion site (FIG. 10E) as well as in residual white matter (FIG. 10F). To assess whether GGF2 treatment influenced p75+ Schwann cell precursor cells, sections from the injury epicenter at 28 days post injury were stained with an antibody to p75. It was shown that GGF2 treatment does not significantly affect the number of p75+ Schwann precursor cells in non-white matter near the injury epicenter at 28 days post injury (FIG. 13).

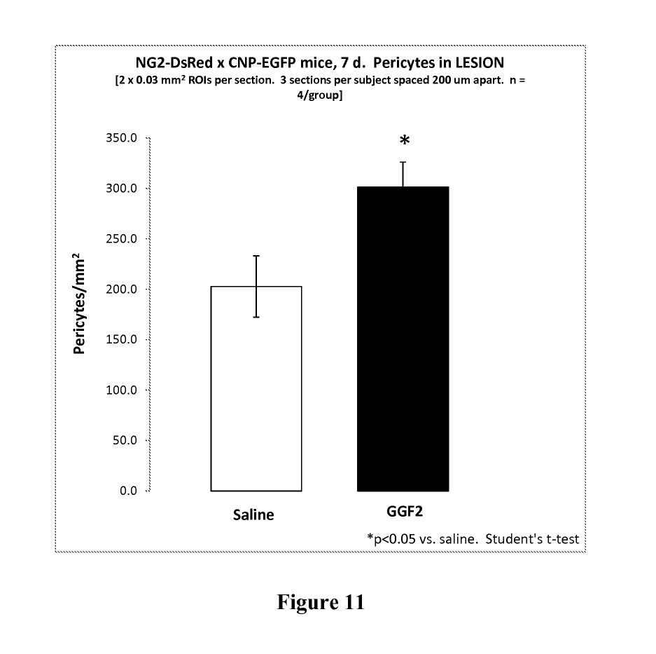

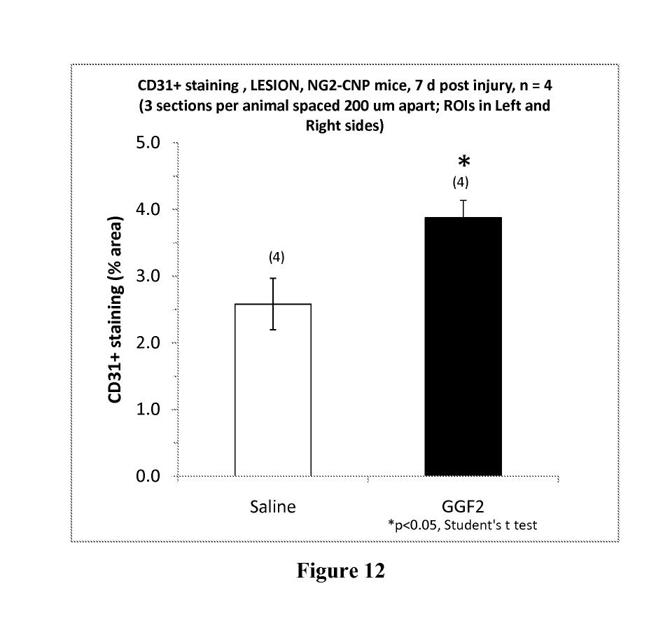

To assess whether GGF2 treatment influenced pericyte production or CD31 production, sections from NG2-dsRed×CNP-EGFP double transgenic mice within the lesion were labeled with antibodies against markers for pericytes (antibodies to PDGFRβ) and blood vessels (antibodies to CD31). It was found that GGF2 treatment increased the numbers of pericytes and CD31+ staining at 7 days post injury (FIGS. 11 and 12). An increase in CD31+ staining is suggestive of revascularization due to GGF2 treatment. Pericyte and CD31+ staining was also carried out at the lesion border and in spared ventrolateral white matter (VLWM), and it was found that GGF2 treatment had no effect on pericyte number or CD31+ staining in these regions.

Systemic administration of GGF2 stimulates proliferation of NG2⁺ cells in the injured spinal cord, increases oligodendrogenesis and myelination in residual tissue, and significantly improves functional recovery. This treatment strategy, based on stimulating endogenous recovery mechanisms, provides beneficial effects even though treatment is initiated a full 24 hours after injury.

GGF2 represents soluble NRG1 Type II, a compound that enhances myelination in vitro at subnanomolar concentrations in Schwann cell-DRG co-cultures. GGF2 treatment also enhances remyelination in vivo in a mouse model of multiple sclerosis, improves functional recovery from peripheral nerve crush in rats, and attenuates free radical release from activated microglial cells in vitro. GGF2 is a known mitogen for Schwann cells, oligodendrocytes, and oligodendrocyte progenitors derived from injured spinal cord in vitro. However, present results are the first to show that GGF2 treatment enhances oligodendrogenesis and myelination in vivo in adult rats and mice after SCI.

As rat and mouse models of contusion SCI have most of the tissue loss at the injury epicenter, including loss of neurons, axons and glia by 24 hours after injury, GGF2 treatment beginning at 24 hours after SCI would act on recovery processes that are activated after SCI. Multiple beneficial effects of GGF2 treatment were evident chronically at 4-6 weeks after SCI, but there was no effect of drug treatment on tissue preservation or hindlimb function at 1 week after injury. However, GGF2 treatment significantly increased the acute proliferation of NG2-expressing cells, a potential source for replacement of lost glia after SCI.

The level of endogenous proliferation of NG2⁺ cells is not saturated in the first week after injury, and can be further increased by daily treatment with exogenous GGF2 beginning 1 day post injury. No effect of GGF2 treatment on proliferation of OX42⁺ microglia/macrophages in spared WM of the injured rat spinal cord was detected, however.

The GGF2 induced increase in acute proliferation of NG2⁺ cells after SCI could ultimately lead to improved functional recovery via increased oligodendrogenesis and improved remyelination of spared axons chronically. Myelinated white matter increased chronically after injury in the rat model of contusive SCI, an effect that was associated with significantly improved hind limb function. Stimulating endogenous OPCs, thus, provides beneficial effects.

Unbiased stereology determined the total number of oligodendrocytes in spinal cord tissue at and adjacent to the injury site at 28 days post injury. GGF2 treated subjects had a significantly higher number of mature oligodendrocytes at the injury site chronically after SCI than saline treated controls, and this effect was associated with significantly improved hind limb locomotion. However, no change in myelinated residual white matter area was detected chronically after SCI in the mouse. Immunohistochemical methods used to provide additional measures of the relative number of axons (NF200 staining) and degree of CNS myelination (PLP staining) at the injury site showed no difference between the GGF2-treated and saline control groups. Interestingly, we found increased staining for P0, the major structural protein in PNS myelin, at the injury site in GGF2 treated subjects. These findings suggest that the improved functional recovery observed in mice was associated with improved myelination by Schwann cells.

Considerable P0⁺ Schwann cell myelin staining was detected at the injury epicenter, particularly at dorsal root entry zones. P0 staining was also seen at ventral root entry zones and within the lesion (FIG. 10). Co-labeling experiments with the axonal marker NF200 showed evidence of P0 myelination of surviving axons. Importantly, GGF2 treatment resulted in increased P0 expression at the injury epicenter that was associated with improved locomotor function.

Although no increase in CNS myelination was detected in the SCI mice treated with GGF2 by eriochrome staining and immunohistochemical labeling of CNS myelin (PLP) these methods may not distinguish between intact myelin and myelin debris that could potentially obscure an effect of GGF2 treatment on newly formed myelin. Alternatively, or in addition, the significantly increased oligodendrogenesis in the treated mice could contribute to enhanced functional recovery through mechanisms other than remyelination. CNS neurons require multiple signals for optimal survival and maturation, and continued oligodendrocyte-derived signals are necessary to maintain neuronal integrity. In addition to their role in myelinating axons, oligodendrocytes release soluble factors including insulin like growth factor (IGF-1), glial derived neurotrophic factor (GDNF) and brain derived neurotrophic factor (BDNF) that can promote neuronal survival, maintain axonal structure and support synaptic plasticity in surviving axons. Furthermore, NRG signaling influences the release of factors including BDNF and NT-3 from glial cells to promote neuronal survival and synapse formation. The expression and release of trophic factors represents a mechanism by which oligodendrocytes interact with neurons to form and maintain functional neural circuits in the injured spinal cord. GGF2 treatment may enhance the ability of oligodendrocytes to carry out such functions and enhance functional recovery.

An intriguing novel finding of this mouse SCI study is that GGF2 treatment up-regulated the expression of Sox2 in oligodendrocyte lineage cells. Sox2 (Sex determining region of Y chromosome (SRy)-related high mobility group box2) is one of the earliest transcription factors expressed in the CNS. It plays an important role in maintaining the proliferative and undifferentiated state of neural stem cells, and its expression is downregulated upon differentiation to neurons and OPCs. Interestingly, purified rat OPCs treated with bone morphogenic protein 2 (BMP2) are converted to multipotent stem-like cells through a process which is dependent upon the re-activation of the Sox2 gene. Further, astrocytes re-express Sox2 upon re-entry into the cell cycle in vivo after injury in the mouse cortex. Sox2 is expressed early in the Schwann cell lineage, down-regulated upon differentiation, and is rapidly re-expressed upon injury. Its expression is implicated in Schwann cell de-differentiation after peripheral nerve injury. Sox2-expressing cell numbers increase dramatically in the spared WM of spinal cord after contusion injury. The current study in CNP-EGFP mice shows that GGF2 treatment significantly increases the number of Sox2-expressing oligodendrocyte-lineage cells at 7 days after SCI compared to that in saline treated controls (FIGS. 7B and 7C). Given that OPCs do not usually express Sox2, these EGFP⁺/Sox2⁺ cells may reflect oligodendrocyte lineage cells at various stages of development that, after injury, have converted to a more stem-like state.

GGF2 treatment also significantly increased the number of non-oligodendrocyte lineage NG2 cells (EGFP⁻/NG2⁺) in ventral lateral white matter (VLWM) at the injury epicenter at 7 days post injury (FIG. 6E). The pericyte is a cell type that expresses NG2 and is located at the abluminal surface of endothelial cells of capillaries, arterioles and venules. Pericytes play a major role in angiogenesis as well as the development and maintenance of BBB tight junctions. Therapeutic interventions that enhance the response of these cells after SCI could be of significant benefit in re-vascularization of the injury site. Furthermore, numerous studies have provided evidence for the multipotential stem cell characteristics of pericytes, suggesting that pericytes represent a source of adult stem cells. As stated above, it was found that GGF2 treatment increases the number of pericytes with the lesion site at 7 days post spinal cord injury (FIG. 11).

In conclusion, daily systemic GGF2 treatment in the week following SCI enhances functional recovery in both rat and mouse models of clinically relevant contusive SCI. The improved recovery was associated with an increase in NG2⁺ cells by one week and increased oligodendrogenesis and myelination chronically after injury. This study achieved a beneficial functional outcome with treatment beginning at 24 hours post injury.

What is claimed is:

1. A method of treating spinal cord injury in a subject, comprising administering at least one dosage of less than 1 mg/kg of GGF2 to the subject.

2. The method of claim 1, further comprising administering a plurality of dosages each dosage comprising less than 1 mg/kg of GGF2 to the subject.

3. The method of claim 1, wherein the first dose is administered to the subject at least one day following a spinal cord injury.

4. The method of claim 2, wherein each dosage is administered on a different day.

5. A method of promoting proliferation of neural stem cells comprising contacting the neural stem cells with GGF2.

6. The method of claim 5, wherein the contacting step is performed multiple times.

7. The method of claim 6, wherein the contacting step is performed daily for seven days.

8. The method of claim 6, further comprising performing the contacting step at least weekly for two, three, or four weeks.

9. The method of claim 5, wherein the contacting step is performed in vitro.

10. The method of claim 5, wherein the contacting step is performed in vivo.

11. The method of claim 10, wherein the in vivo contacting step is performed within one day of a central nervous system injury.

12. The method of claim 5, further comprising contacting the neural stem cells with FGF2.

13. A method of promoting revascularization of neural tissue following central nervous system injury in a subject comprising administering to the subject GGF2.

14. The method of claim 13, wherein the administration occurs within one day of injury.

15. The method of claim 13, wherein the administration is in multiple doses.

16. The method of claim 15, wherein the GGF2 is administered daily for seven days.

17. The method of claim 16, wherein the GGF2 is administered weekly for two, three, or four weeks.

18. The method of claim 13, further comprising administering FGF2 to the subject.
